(19) United States
(12) Patent Application Publication
(10) Pub. No.: US 2021/0298740 A1
(43) Pub. Date: Sep. 30, 2021

(54) SYNDESMOSIS INSERTION CONSTRUCT

(71) Applicant: In2Bones USA, LLC, Memphis, TN (US)

(72) Inventors: Alan G. Taylor, Memphis, TN (US); Rebecca Hawkins Wahl, Escondido, CA (US); Bradley A.S. Cowan, Memphis, TN (US)

(73) Assignee: In2Bones USA, LLC, Memphis, TN (US)

(21) Appl. No.: 17/112,767

(22) Filed: Dec. 4, 2020

Related U.S. Application Data
(63) Continuation-in-part of application No. 16/854,264, filed on Apr. 21, 2020.
(60) Provisional application No. 62/994,922, filed on Mar. 26, 2020.

(51) Int. Cl. A61B 17/04

(52) U.S. Cl. CPC A61B 17/0401; A61B 2017/0445; A61B 2017/0403; A61B 2017/0409

(57) ABSTRACT

An apparatus and methods are provided for a syndesmosis treatment assembly that includes an insertion assembly and a syndesmosis suture construct for securing a first bone against a second bone. The syndesmosis suture construct is configured to be passed through a bone hole drilled across the first bone and the second bone and cinched to press the bones together. In an embodiment, the insertion assembly includes a sleeve and a push rod for inserting the syndesmosis suture construct through the bone hole. The sleeve maintains the syndesmosis suture construct in an elongate configuration suitable for being inserted through the bone hole. The push rod enables deploying a distal fixator comprising the syndesmosis suture construct to be anchored against a distal contact surface of the second bone. The sleeve and push rod are removable from the bone hole before the syndesmosis suture construct is cinched to secure the bones.

### PRIORITY

This application is a continuation-in-part of, and claims the benefit of, U.S. patent application, entitled “Syndesmosis Insertion Construct,” filed on Apr. 21, 2020, and having application Ser. No. 16/854,264, which claims the benefit of, and priority to, U.S. Provisional application, entitled “Syndesmosis Insertion Construct,” filed on Mar. 26, 2020 and having application Ser. No. 62/994,922, the entirety of said applications being incorporated herein by reference.

### FIELD

Embodiments of the present disclosure generally relate to the field of securing bones together. More specifically, embodiments of the disclosure relate to systems and methods for a syndesmosis treatment assembly that includes an insertion assembly and a syndesmosis suture construct for securing a first bone against a second bone.

### BACKGROUND

A syndesmosis is a slightly movable fibrous joint in which bones such as the tibia and fibula are joined together by connective tissue. The distal tibia fibular joint and the radioulnar joint are examples of syndesmoses. Injuries to the ankle syndesmosis are common and frequently occur in patients having ankle fractures.

Due to the complex biomechanics of ankle syndesmosis injuries and the relatively increased healing time associated with them, there has been widespread debate on both the strongest and most appropriate methods of fixation when treated operatively. Conventional treatments for ankle syndesmosis injuries include metallic or bioabsorbable screw fixation, as well as various methods of suture button fixation. A benefit of suture button fixation treatments is that they generally do not require additional procedures for removal of implants as do screw fixation treatments.

There is an ongoing need for the development of bone fusion capabilities such as that related to, for example, treating injuries to the ankle syndesmosis. Provided herein are embodiments and methods for a syndesmosis treatment assembly that includes an insertion assembly and a syndesmosis suture construct that is configured to cinch a first bone against a second bone.

### SUMMARY

An apparatus and methods are provided for a syndesmosis treatment assembly that includes an insertion assembly and a syndesmosis suture construct for securing a first bone against a second bone. The syndesmosis suture construct is configured to be passed through a bone hole drilled across the first bone and the second bone and placed into a cinched configuration that presses the first bone against the second bone. The insertion assembly includes a sleeve and a push rod that facilitate inserting the syndesmosis suture construct through the bone hole. The sleeve maintains the syndesmosis suture construct with the push rod in an elongate configuration suitable for being inserted through the bone hole. The push rod is configured to enable deploying a distal fixator comprising the syndesmosis suture construct such that the distal fixator may be anchored against a distal contact surface of the second bone. The sleeve and the push rod are configured to be removed from the bone hole before the syndesmosis suture construct is cinched to secure the first bone against the second bone.

In an exemplary embodiment, a syndesmosis treatment assembly comprises: a syndesmosis suture construct for securing a first bone against a second bone; and an insertion assembly for implanting the syndesmosis suture construct in a patient.

In another exemplary embodiment, the syndesmosis suture construct is configured to be passed through a bone hole drilled across the first bone and the second bone and placed into a cinched configuration that presses the first bone against the second bone. In another exemplary embodiment, the insertion assembly includes a sleeve and a push rod that are configured to facilitate inserting the syndesmosis suture construct through the bone hole. In another exemplary embodiment, the sleeve is configured to retain the syndesmosis suture construct and the push rod during inserting the syndesmosis suture construct through the bone hole. In another exemplary embodiment, the sleeve is configured to maintain the syndesmosis suture construct in an elongate configuration suitable for being inserted into the bone hole. In another exemplary embodiment, the push rod is an elongate member that includes a shaft extending from a distal end to a proximal gripping end and is configured to enable a surgeon to deploy a distal fixator comprising the syndesmosis suture construct. In another exemplary embodiment, the sleeve and the push rod are configured to be removed from the bone hole so as to facilitate the syndesmosis suture construct being cinched to secure the first bone against the second bone.

In an exemplary embodiment, an insertion assembly for implanting a syndesmosis suture construct in a bone hole drilled across a first bone and a second bone of a patient comprises: a sleeve for housing the syndesmosis suture construct in an elongate configuration; and a push rod for implanting and deploying the syndesmosis suture construct.

In another exemplary embodiment, the sleeve comprises a generally elongate hollow tube that includes a slit that extends along the length of the sleeve. In another exemplary embodiment, the slit allows the sleeve to radially constrict so as to hold the syndesmosis suture construct and the push rod in an assembled configuration suitable for implantation into the bone hole.

In another exemplary embodiment, the push rod comprises a generally elongate member including a shaft extending from a distal end to a proximal gripping end. In another exemplary embodiment, the distal end comprises a blunt surface configured for causing a distal fixator of the syndesmosis suture construct to exit the sleeve when the push rod is advanced in a distal direction. In another exemplary embodiment, the push rod includes a boss configured to limit a depth to which the push rod may be advanced into the sleeve.

In an exemplary embodiment, a method for treating a syndesmosis by securing a first bone and a second bone of a patient comprises: drilling a bone hole through the first bone and the second bone; providing a syndesmosis suture construct for inserting into the bone hole cinching the first bone against the second bone; housing the syndesmosis suture construct in an elongate configuration within a sleeve; placing a push rod within the sleeve adjacent to the syndesmosis suture construct; inserting the sleeve through the bone hole until the sleeve reaches a distal contact surface of the second bone; deploying a distal fixator of the syndesmosis suture construct; anchoring the distal fixator against the distal contact surface; withdrawing the push rod and the sleeve from the bone hole; and cinching the syndesmosis suture construct to press the first bone against the second bone.

In another exemplary embodiment, deploying the distal fixator includes advancing the push rod in a distal direction within the sleeve such that a distal end of the push rod causes the distal fixator to exit the sleeve. In another exemplary embodiment, advancing includes advancing the push rod through the sleeve until a boss comprising the push rod contacts the sleeve so as to indicate that the distal fixator is free of the sleeve. In another exemplary embodiment, anchoring the distal fixator includes manipulating one or more suture ends comprising the syndesmosis suture construct to move the distal fixator into contact with the distal contact surface.

In another exemplary embodiment, withdrawing the sleeve includes moving the syndesmosis suture construct through a slit extending along a length of the sleeve while sliding the sleeve out of the bone hole. In another exemplary embodiment, cinching the syndesmosis suture construct includes anchoring a proximal fixator of the syndesmosis suture construct against a proximal contact surface of the first bone. In another exemplary embodiment, cinching the syndesmosis suture construct includes forming one or more surgical knots to secure a cinched configuration of the first bone and the second bone.

In an exemplary embodiment, a syndesmosis treatment assembly comprises: a syndesmosis suture construct for securing a first bone against a second bone; an insertion assembly for implanting the syndesmosis suture construct in a patient; and one or more tensioning handles for cinching the syndesmosis suture construct.

In another exemplary embodiment, the insertion assembly includes a deployment guidewire, a pull wire, a deployment suture, and a proximal deployment suture. In another exemplary embodiment, the deployment guidewire is a generally elongate member that includes a sharpened distal end suitable for being passed through a bone hole drilled into a first bone and a second bone of a patient. In another exemplary embodiment, the pull wire is attached to a proximal end of the deployment guidewire and coupled with a distal fixator comprising the syndesmosis suture construct. In another exemplary embodiment, the deployment guidewire, the pull wire, and the proximal deployment suture may be used to maintain the syndesmosis suture construct in an elongate configuration suitable for being inserted into the bone hole drilled. In another exemplary embodiment, the deployment suture is configured to enable a surgeon to cause the distal fixator of the syndesmosis suture construct to contact a far cortex of the second bone.

In an exemplary embodiment, an insertion assembly for implanting a syndesmosis suture construct in a bone hole drilled across a first bone and a second bone of a patient comprises: a deployment guidewire; a pull wire; a deployment suture; and a proximal deployment suture.

In another exemplary embodiment, the deployment guidewire is configured for inserting the syndesmosis suture construct into the bone hole. In another exemplary embodiment, the deployment guidewire includes a sharpened distal end for being passed through skin adjacent to a far cortex of the second bone. In another exemplary embodiment, the pull wire is configured for drawing a distal fixator comprising the syndesmosis suture construct through the bone hole. In another exemplary embodiment, the deployment suture is configured for deploying the distal fixator against a far cortex of the second bone. In another exemplary embodiment, the proximal deployment suture is configured for maintaining the syndesmosis suture construct in an elongate configuration suitable for being inserted into the bone hole.

In another exemplary embodiment, the insertion assembly further comprises one or more tensioning handles configured to facilitate pulling suture ends comprising the syndesmosis suture construct during cinching the first bone and the second bone. In another exemplary embodiment, the one or more tensioning handles each comprises a generally elongate member having suture grooves disposed along a length of the tensioning handle. In another exemplary embodiment, the sutures grooves are each configured to fixedly receive a portion of suture such that a surgeon may grasp the tensioning handle during pulling the suture.

In an exemplary embodiment, a method for treating a syndesmosis by securing a first bone and a second bone of a patient comprises: drilling a bone hole through the first bone and the second bone; providing a syndesmosis suture construct for inserting into the bone hole and cinching the first bone against the second bone; pulling the syndesmosis suture construct through the bone hole by way of a deployment guidewire and a pull wire; deploying a distal fixator of the syndesmosis suture construct by way of a deployment suture; maintaining tension on the syndesmosis suture construct by way of a proximal deployment suture; cinching the syndesmosis suture construct to press the first bone against the second bone; and forming one or more surgical knots to secure a cinched configuration of the first bone and the second bone.

In another exemplary embodiment, drilling includes using a first cannulated bone drill to form a first bone hole through the first bone and then using a second cannulated bone drill to form a second bone hole through the second bone, the first cannulated bone drill having a larger diameter than a diameter of the second cannulated bone drill. In another exemplary embodiment, pulling the syndesmosis suture construct includes passing the deployment guidewire and the pull wire through skin near a far cortex of the second bone. In another exemplary embodiment, deploying includes using the deployment suture to cause the distal fixator to contact the far cortex of the second bone. In another exemplary embodiment, deploying includes removing the deployment guidewire, the pull wire, and the deployment suture after causing the distal fixator to contact the far cortex of the second bone.

### BRIEF DESCRIPTION OF THE DRAWINGS

The drawings refer to embodiments of the present disclosure in which:

FIG. 1 illustrates an isometric exploded view of an exemplary embodiment of a syndesmosis treatment assembly that includes a syndesmosis suture construct and an insertion assembly, in accordance with the present disclosure;

FIG. 2 illustrates an isometric view of the syndesmosis treatment assembly of FIG. 1 wherein the syndesmosis suture construct is loaded into the insertion assembly;

FIG. 3 illustrates an isometric view of an exemplary embodiment of a sleeve comprising the insertion assembly of FIGS. 1-2;

FIG. 4 illustrates an isometric view of a push rod comprising the insertion assembly of FIGS. 1-2;

FIG. 5 illustrates a side view of a distal end of the syndesmosis treatment assembly of FIG. 2 in absence of the sleeve, wherein an exemplary embodiment of a distal fixator comprising the syndesmosis suture construct of FIG. 1 is in contact with a distal end of the push rod of FIG. 4;

FIG. 6 illustrates a side view of the syndesmosis treatment assembly of FIG. 2 including an exemplary embodiment of a restrictor configured to retain a distal fixator of the syndesmosis suture construct housed within a distal end of the insertion assembly;

FIG. 7 illustrates a side ghost-view of an exemplary-use environment wherein the syndesmosis treatment assembly of FIG. 2 is aligned with a bone hole drilled through a first bone and a second bone to be cinched together;

FIG. 8 illustrates an isometric view of a distal fixator comprising the syndesmosis treatment assembly of FIG. 2 being inserted into a proximal opening of the bone hole of FIG. 7;

FIG. 9 illustrates an isometric view of the exemplary-use environment of FIG. 7 wherein the syndesmosis treatment assembly extends through the bone hole into the second bone;

FIG. 10 illustrates the exemplary-use environment of FIG. 7 wherein the syndesmosis treatment assembly extends through the bone hole such that the distal fixator extends through a distal contact surface of the second bone;

FIG. 11 illustrates the exemplary-use environment of FIG. 10 wherein the distal fixator is placed into contact with the distal contact surface of the second bone;

FIG. 12 illustrates the exemplary-use environment of FIG. 11 wherein the push rod has been withdrawn from the insertion assembly;

FIG. 13 illustrates the exemplary-use environment of FIG. 12 wherein the sleeve has been withdrawn from the bone hole and a proximal fixator of the syndesmosis suture contract has been cinched against a proximal contact surface of the first bone, according to the present disclosure;

FIG. 14 illustrates an isometric view of an exemplary embodiment of a syndesmosis treatment assembly that is configured to cinch a first bone against a second bone, according to the present disclosure;

FIG. 15 illustrates an exemplary-use environment wherein an exemplary embodiment of a wire guide is being used to direct a guidewire being inserted into a first bone and a second bone;

FIG. 16 illustrates an exemplary-use environment wherein a distal end of the wire guide of FIG. 15 is being engaged with a bone fusion plate;

FIG. 17 illustrates an exemplary-use environment wherein an exemplary embodiment of a wire guide is being withdrawn from a guidewire that is inserted across a first bone and a second bone;

FIG. 18 illustrates an exemplary-use environment wherein a first cannulated bone drill is being moved along a guidewire to a near cortex of a first bone to be drilled;

FIG. 19 illustrates the exemplary-use environment of FIG. 18 wherein the first cannulated bone drill is being withdrawn after drilling a first bone hole that breaches a far cortex of the first bone;

FIG. 20 illustrates the exemplary-use environment of FIG. 19 wherein a second cannulated bone drill is being moved along the guidewire through the first bone hole to a near cortex of a second bone to be drilled;

FIG. 21 illustrates the exemplary-use environment of FIG. 20 wherein the second cannulated bone drill is being withdrawn after drilling a second bone hole that breaches a far cortex of the second bone;

FIG. 22 illustrates a side ghost-view of an exemplary-use environment wherein the syndesmosis treatment assembly of FIG. 14 is aligned with a bone hole drilled through a first bone and a second bone to be cinched together;

FIG. 23 illustrates the exemplary-use environment of FIG. 22 wherein the syndesmosis treatment assembly extends through the bone hole such that a distal fixator of the syndesmosis treatment assembly extends through a far cortex of the second bone;

FIG. 24 illustrates the exemplary-use environment of FIG. 23 wherein the distal fixator is placed into contact with the far cortex of the second bone;

FIG. 25 illustrates the exemplary-use environment of FIG. 24 wherein tension is being maintained on a proximal deployment suture while the first bone is being cinched against the second bone;

FIG. 26 illustrates the exemplary-use environment of FIG. 25 wherein a proximal fixator of the syndesmosis treatment assembly is seated within a proximal opening of the bone hole during cinching the first bone against the second bone;

FIG. 27 illustrates the exemplary-use environment of FIG. 26 wherein the proximal deployment suture has been removed from the proximal fixator after the first and second bones have been desirably cinched together;

FIG. 28 illustrates an exemplary embodiment of a pair of tensioning handles that are configured to facilitate pulling sutures ends during cinching a first bone and a second bone together;

FIG. 29 illustrates an exemplary embodiment of a syndesmosis implant kit that is configured for cinching a first bone against a second bone; and

FIG. 30 illustrates an exemplary embodiment of a syndesmosis reamer kit comprising a first cannulated bone drill and a second cannulated bone drill configured for drilling a bone hole across a first bone and a second bone to be cinched together according to the present disclosure.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first suture,” may be made. However, the specific numeric reference should not be interpreted as a literal sequential order but rather interpreted that the “first suture” is different than a “second suture.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

A syndesmosis is a slightly movable fibrous joint in which bones such as the tibia and fibula are joined together by connective tissue. The distal tibia fibular joint and the radioulnar joint are examples of syndesmoses. Injuries to the ankle syndesmosis are common and frequently occur in patients having ankle fractures. There is an ongoing need for the development of bone fusion capabilities such as that found in, for example, treating injuries to the ankle syndesmosis. Provided herein are embodiments and methods for a syndesmosis treatment assembly that includes an insertion assembly and a syndesmosis suture construct configured to be placed into a cinched configuration that presses a first bone against a second bone.

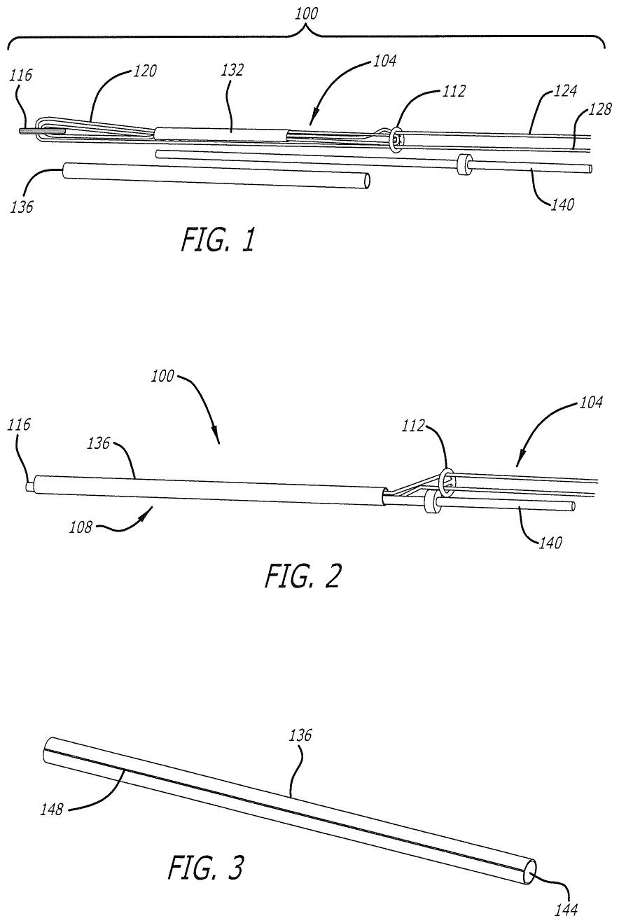

FIG. 1 illustrates an exemplary embodiment of a syndesmosis treatment assembly 100 that is configured to cinch a first bone against a second bone, according to the present disclosure. The syndesmosis treatment assembly 100 of FIG. 1 includes a syndesmosis suture construct 104 and an insertion assembly 108 that facilitates implanting the syndesmosis suture construct 104 in a patient.

In general, embodiments of the syndesmosis suture construct 104 are configured to be passed through a bone hole drilled across the first bone and the second bone and placed into a cinched configuration that presses the first bone and the second bone together. In the embodiment illustrated in FIG. 1, the syndesmosis suture construct 104 comprises a proximal fixator 112 that is configured to contact the first bone, and a distal fixator 116 that is configured to contact the second bone. As shown, the proximal fixator 112 is generally a round, button-shaped member suitable for contacting bone, and the distal fixator 116 is an oblong-shaped member suitable for contacting bone. As will be appreciated, the oblong-shape of the distal fixator 116 facilitates passing, or drawing, the distal fixator 112 through a bone hole drilled in the bones to be treated. It is contemplated, however, that the proximal and distal fixators 112, 116 may include any of various shapes that are found to be advantageous for pressing bones together, without limitation.

As shown in FIG. 1, a suture 120 is looped through the proximal fixator 112 and the distal fixator 116 such that opposite suture ends 124, 128 extend from the proximal fixator 112. The suture 120 includes a splice 132 is configured to slidably ride on portions of the suture 120 extending between the proximal and distal fixators 112, 116. The opposite suture ends 124, 128 extending from the proximal fixator 112 facilitate a practitioner, such as a surgeon, pulling on the suture 120 to place the syndesmosis suture construct 104 into a cinched configuration suitable for pressing bones together. As will be appreciated, the suture 120 generally may be comprised of any of various suture materials that are suitable for syndesmosis treatment. Further, it should be understood that since the splice 132 is to be positioned inside the bone hole, a diameter of the splice 132 preferably is less than the diameter of the bone hole. Further details pertaining to the syndesmosis suture construct 104, as well as operation thereof, can be found in U.S. patent application, entitled “Syndesmosis Treatment Construct,” filed on Dec. 17, 2019 and having Ser. No. 16/717,981, the entirety of which is incorporated herein by reference and made a part of application.

With continuing reference to FIG. 1, the insertion assembly 108 includes a sleeve 136 and a push rod 140 that are configured to facilitate inserting the syndesmosis suture construct 104 into a patient. As best shown in FIG. 2, the syndesmosis suture construct 104 and the push rod 140 may be retained within the sleeve 136 during inserting the syndesmosis suture construct 104 into the patient. In an assembled configuration, shown in FIG. 2, the sleeve 136 maintains the syndesmosis suture construct 104 in an elongate configuration suitable for being inserted into a bone hole drilled into a first bone and a second bone of the patient. The push rod 140 enables the surgeon to deploy the distal fixator 116 of the syndesmosis suture construct 104 as described herein.

FIG. 3 illustrates an exemplary embodiment of the sleeve 136 comprising the insertion assembly 108 shown in FIGS. 1-2. The sleeve 136 is a generally elongate hollow tube that includes a longitudinal opening 144 and a slit 148 that extend along the length of the sleeve 136. The longitudinal opening 144 has a diameter suitable for receiving the syndesmosis suture construct 104 and the push rod 140 into the sleeve 136. The slit 148 allows the sleeve 136 to radially constrict so as to hold the syndesmosis suture construct 104 and the push rod 140 in the assembled configuration shown in FIG. 2. As such, the sleeve 136 is configured to secure and hold the syndesmosis suture construct 104 and the push rod 140 together during implantation into a bone hole drilled across a first one and a second bone.

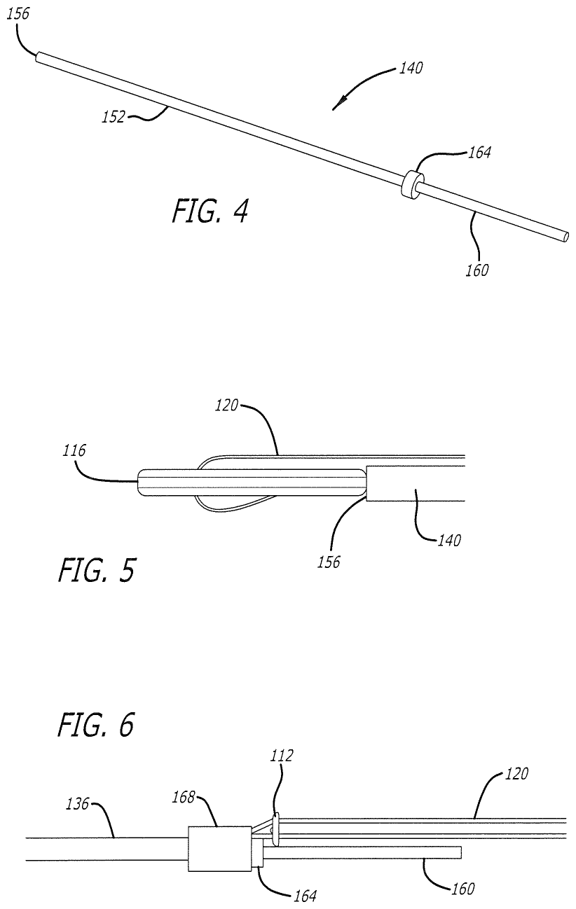

FIG. 4 illustrates an exemplary embodiment of the push rod 140 comprising the insertion assembly 108 shown in FIGS. 1-2. The push rod 140 is a generally elongate member including a shaft 152 extending from a distal end 156 to a proximal gripping end 160. The gripping end 160 is adapted for being grasped in a hand of the surgeon during operation of the syndesmosis treatment assembly 100. The shaft 152 is configured to sit adjacent to the syndesmosis suture construct 104 within the sleeve 136 and to keep the syndesmosis suture construct 104 in the elongate configuration discussed with respect to FIG. 2. The shaft 152 includes a smooth surface suitable for contacting and sliding adjacent to the suture 120 without fraying or otherwise damaging the suture 120.

As shown in FIG. 5, the distal end 156 comprises a blunt surface configured for contacting the distal fixator 116. As such, the distal end 156 is configured to cause the distal fixator 116 to exit the sleeve 136 when the surgeon advances the push rod 140 in a distal direction. As best shown in FIG. 2, the shaft 152 generally includes a length that is greater than the length of the sleeve 136 and thus is suitable for pushing distal fixator 116 out of the sleeve 136. A boss 164 disposed between the shaft 152 and the gripping end 160, as shown in FIG. 4, is configured to limit the depth to which the shaft 152 may be inserted into to the sleeve 136. To this end, the boss 164 generally includes a diameter that is greater than the diameter of the sleeve 136. It is contemplated that the boss 164 is to be disposed at a position along the push rod 140 such that the distal fixator 116 completely exits a distal end of the sleeve 136 when the boss 164 contacts a proximal end of the sleeve 136. Thus, contact between the boss 164 and the sleeve 136 may serve to indicate to the surgeon that full deployment of the distal fixator 116 has occurred.

As will be appreciated, an undesirable deployment of the distal fixator 116, such as due to inadvertently advancing the push rod 140 within the sleeve 136 while inserting the syndesmosis treatment assembly 100 into the bone hole, may complicate performing the syndesmosis treatment and cause damage to one or both of the first and second bones. It is contemplated, therefore, that inhibiting unintended movement of the push rod 140 within the sleeve 136 may advantageously reduce instances of complications arising during syndesmosis treatments. To this end, a restrictor 168 may be disposed between the boss 164 and the sleeve 136 as shown in FIG. 6. The restrictor 168 generally is a tube-shaped member configured to retain the distal fixator 116 housed within the sleeve 136 during advancing the syndesmosis treatment assembly 100 into the bone hole. Further, it is contemplated that a slit (not shown) may extend along the length of the restrictor 168 to enable removal of the restrictor 168 from the syndesmosis treatment assembly 100 during performing the syndesmosis treatment, as described herein.

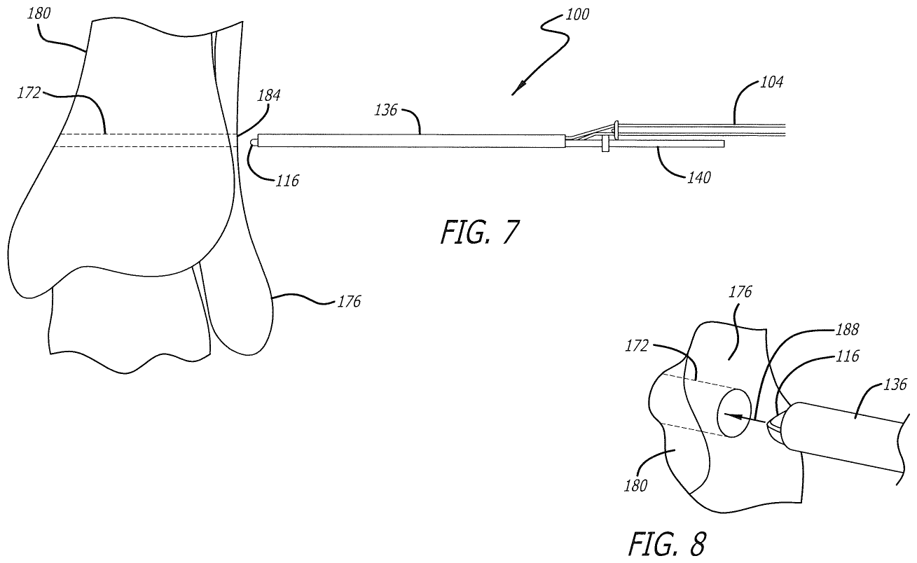

FIG. 7 illustrates an exemplary-use environment wherein the syndesmosis treatment assembly 100, shown in FIG. 2, is aligned with a bone hole 172 in preparation for being inserted into the bone hole 172 during a syndesmosis treatment. The bone hole 172 is drilled through a first bone 176 and a second bone 180 that are to be cinched together by way of the syndesmosis suture construct 104 as discussed herein. As shown in FIG. 7, the syndesmosis suture construct 104 and the push rod 140 are housed, side-by-side, within the sleeve 136, such that the distal fixator 116 may be advantageously inserted through the bone hole 172. As will be appreciated, the bone hole 172 preferably has a diameter that is greater than the diameter of the sleeve 136 so as to facilitate extending the syndesmosis treatment assembly 100 through the bone hole 172.

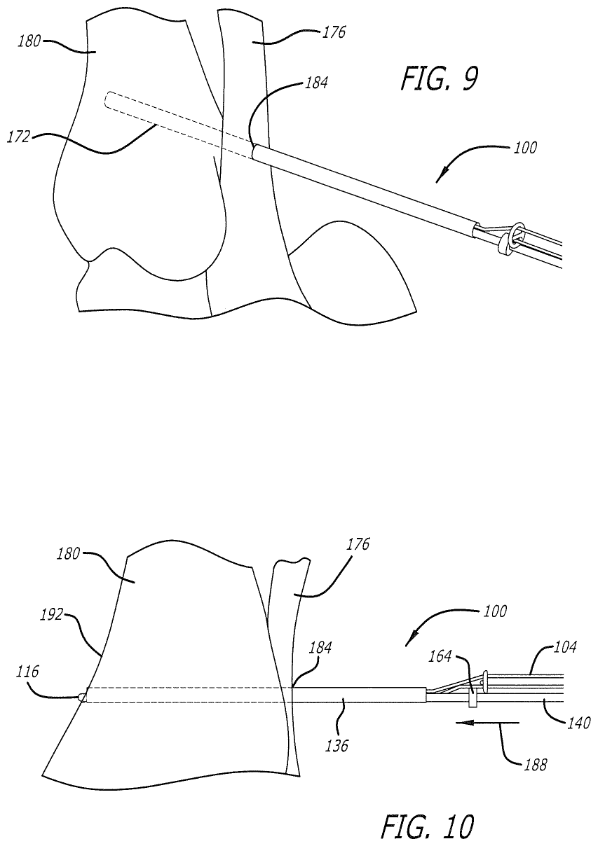

As best shown in FIG. 8, the syndesmosis treatment assembly 100 may be extended through the bone hole 172 by first inserting the portion of the sleeve 136 retaining the distal fixator 116 into a proximal opening 184 of the bone hole 172 and then advancing the syndesmosis treatment assembly 100 in a distal direction 188. The syndesmosis treatment assembly 100 may then be pushed through the portion of the bone hole 172 within the first bone 176 and the portion of the bone hole 172 within the second bone 180 (see FIG. 9) until the sleeve 136 and the distal fixator 116 reaches a distal contact surface 192 of the second bone 180. In some embodiments, any of various suitable markings may be applied to the exterior of the sleeve 136 to indicate a depth within the bone hole 172 to which the syndesmosis treatment assembly 100 must be inserted to cause the distal fixator 116 to and/or slightly beyond the distal contact surface 192 of the second bone 180.

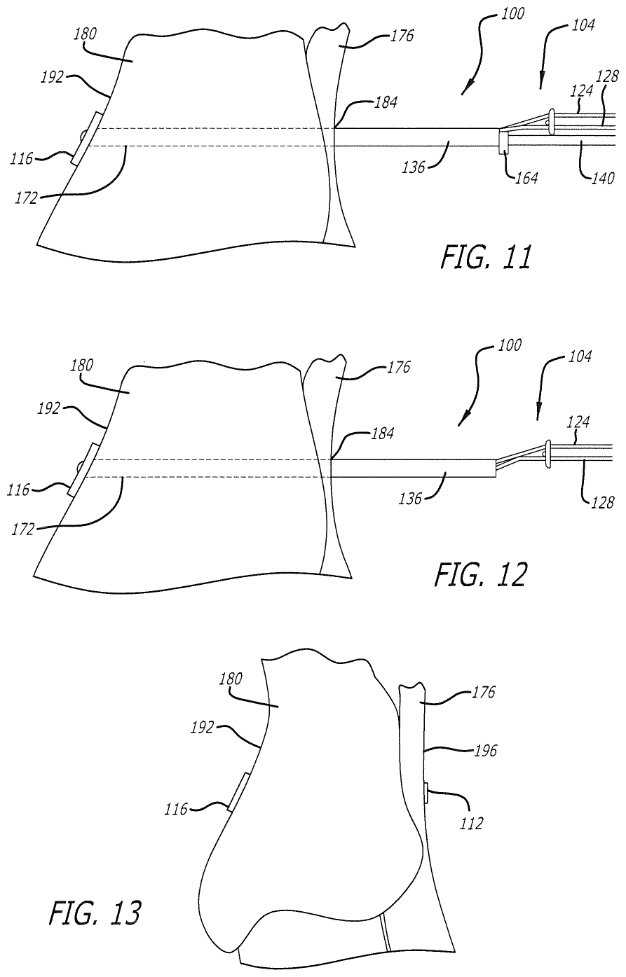

Once the syndesmosis treatment assembly 100 is optimally positioned with respect to the distal contact surface 192, as shown in FIG. 10, the distal fixator 116 may be deployed by advancing the push rod 140 in the distal direction 188 while preventing movement of the sleeve 136. As discussed in connection with FIG. 5, the distal end 156 of the push rod 140 comprises a blunt surface configured to cause the distal fixator 116 to exit the sleeve 136 when the surgeon advances the push rod 140 in the distal direction 188. It is contemplated that the surgeon may advance the push rod 140 in the distal direction 188, through the sleeve 136 until the boss 164 contacts the sleeve 136, as shown in FIG. 11, which indicates that the distal fixator 116 is completely free of the sleeve 136.

After moving the distal fixator 116 out of the sleeve 136, the surgeon may manipulate the suture ends 124, 128 to anchor the distal fixator 116 against the distal contact surface 192 of the second bone 180, as shown in FIG. 11. Those skilled in the art will appreciate that using the sleeve 136 and the push rod 140 to deploy the distal fixator 116 advantageously obviates a need for surgically accessing the distal contact surface 192 of the second bone 180 in an attempt to pull the distal fixator 116 through the bone hole 172. As such, the sleeve 136 and the push rod 140 facilitate anchoring the distal fixator 116 against the distal contact surface 192 by accessing the bone hole 172 solely through the proximal opening 184.

Once the distal fixator 116 is optimally anchored against the distal contact surface 192 of the second bone 180, the push rod 140 may be withdrawn from the sleeve 136, as shown in FIG. 12, leaving only the syndesmosis suture construct 104 disposed within the sleeve 136. Next, the sleeve 136 may be withdrawn from the bone hole 172. It is contemplated that the slit 148, discussed with respect to FIG. 3, enables the surgeon to move the syndesmosis suture construct 104 outside the sleeve 136 while sliding the sleeve 136 out of the bone hole 172. The slit 148, therefore, facilitates removing the syndesmosis suture construct 104 from the sleeve 136 without necessitating prior removal of the proximal fixator 112 from the syndesmosis suture construct 104. As such, the sleeve 136 and the push rod 140 may be utilized with syndesmosis suture constructs, other than the syndesmosis suture construct 104, that include irremovable proximal fixators, without limitation.

Turning now to FIG. 13, once the sleeve 136 is withdrawn from the bone hole 172 and removed from the syndesmosis suture construct 104, as discussed in connection with FIG. 12, the proximal fixator 112 may be anchored against a proximal contact surface 196 of the first bone 176. As shown in FIG. 13, the suture ends 124, 128 (see FIG. 12) have been pulled to tighten the syndesmosis suture construct 104 within the bone hole 172 and thus cinch the first bone 176 against the second bone 180, as desired. During cinching the syndesmosis suture construct 104, the proximal fixator 112 seats within the proximal opening 184, as shown in FIG. 13. Further, in some embodiments, a bone fusion plate may be disposed between the proximal fixator 112 and the proximal contact surface 196 to provide support for the proximal fixator 112. Any one or more of various surgical knots may be formed by the surgeon to ensure that the syndesmosis suture construct 104 maintains a desirably cinched configuration of the first and second bones 176, 180.

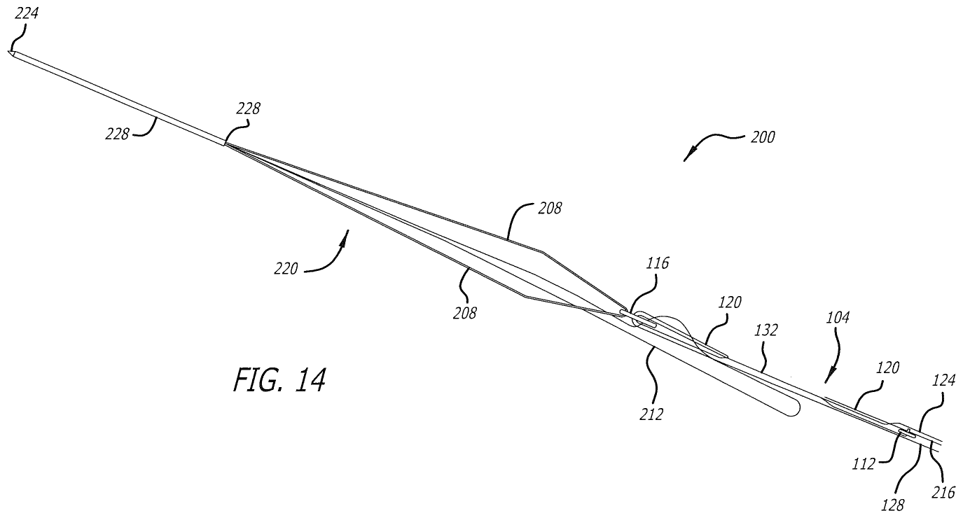

FIG. 14 illustrates an exemplary embodiment of a syndesmosis treatment assembly 200 that is configured to cinch a first bone against a second bone, according to the present disclosure. The syndesmosis treatment assembly 200 of FIG. 14 includes a syndesmosis suture construct 104 that is coupled with a deployment guidewire 204 by way of a pull wire 208 and a deployment suture 212. The syndesmosis suture construct 104 shown in FIG. 14 is substantially identical to the syndesmosis suture construct 104 of FIG. 1, which the exception that a proximal deploying suture 216 is coupled with a proximal fixator 112 of the syndesmosis suture construct 104 and the pull wire 208 and deployment suture 212 are coupled with a distal fixator 116 of the syndesmosis suture construct 104.

As described with respect to FIG. 1, the syndesmosis suture construct 104 of FIG. 14 includes a suture 120 that is looped through the proximal fixator 112 and the distal fixator 116 such that opposite suture ends 124, 128 extend from the proximal fixator 112. The suture 120 includes a splice 132 is configured to slidably ride on portions of the suture 120 extending between the proximal and distal fixators 112, 116. The opposite suture ends 124, 128 extending from the proximal fixator 112 facilitate a practitioner, such as a surgeon, pulling on the suture 120 to place the syndesmosis suture construct 104 into a cinched configuration suitable for pressing bones together. As will be appreciated, the suture 120 generally may be comprised of any of various suture materials that are suitable for syndesmosis treatment. Further, it should be understood that since the splice 132 is to be positioned inside the bone hole, a diameter of the splice 132 preferably is less than the diameter of the bone hole. Further details pertaining to the syndesmosis suture construct 104, as well as operation thereof, can be found in U.S. patent application, entitled “Syndesmosis Treatment Construct,” filed on Dec. 17, 2019 and having Ser. No. 16/717,981, the entirety of which is incorporated herein by reference and made a part of application.

It is contemplated that the deployment guidewire 204, the pull wire 208, the deployment suture 212, and the proximal deployment suture 216 comprise an insertion assembly 220 that advantageously facilitates inserting the syndesmosis suture construct 104 into a patient. The deployment guidewire 204 is a generally elongate member that includes a sharpened distal end 224 suitable for being inserted through a bone hole and advanced through skin at a far cortex of the bone to be treated. The pull wire 208 is attached to a proximal end 228 of the deployment guidewire 204 and coupled with the distal fixator 116. As shown in FIG. 14, the deployment guidewire 204, the pull wire 208, and the proximal deployment suture 216 may be used to maintain the syndesmosis suture construct 104 in an elongate configuration suitable for being inserted into a bone hole drilled into a first bone and a second bone of the patient. The pull wire 208 enables a surgeon to deploy the distal fixator 116 of the syndesmosis suture construct 104 as described herein.

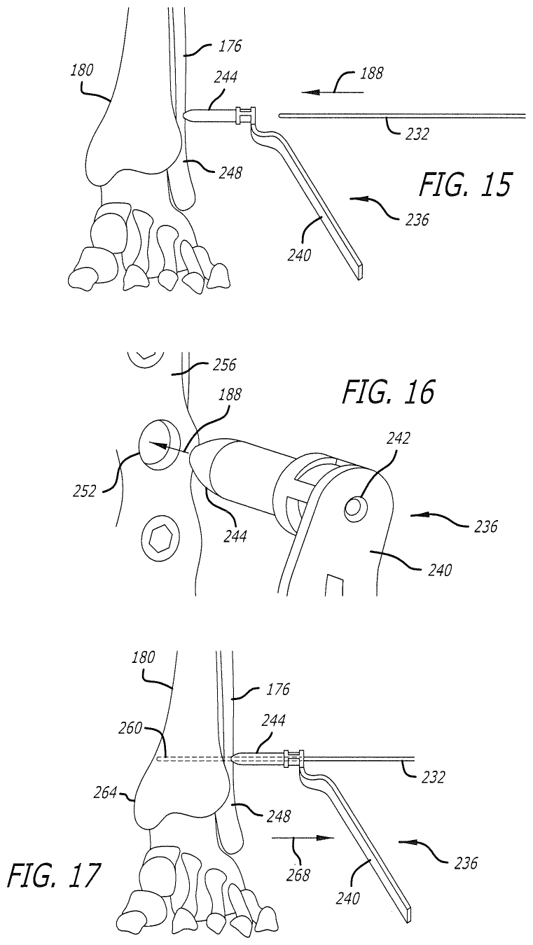

FIGS. 15-21 illustrate an exemplary-use environment wherein a bone hole is being prepared to be drilled across a first bone 176 and a second bone 180 that are to be cinched together by way of the syndesmosis suture construct 104 as discussed herein. As shown in FIG. 15, the bone hole may be prepared by pushing a guidewire 232 across the first and second bones 176, 180 by way of a wire guide 236. The embodiment of the wire guide 236 shown in FIGS. 15-17 includes a handle 240 suitable for grasping the wire guide 236, a cannula 242 for orienting the guidewire 232, and a distal end 244 for supporting the guidewire 232 while being pushed in a distal direction 188 through a near cortex 248 of the first bone 176. It is contemplated that the distal end 244 may be centered into an aperture 252 of a bone fusion plate 256, as shown in FIG. 16, or the distal end 244 may be pressed directly against the first bone 176 in absence of the bone fusion plate 256. As shown in FIG. 17, once an inserted portion 260 of the guidewire 232 extends from the near cortex 248 of the first bone 176 to a far cortex 264 of the second bone 180, the wire guide 236 may be moved from in a proximal direction 268 and removed from the guidewire 232.

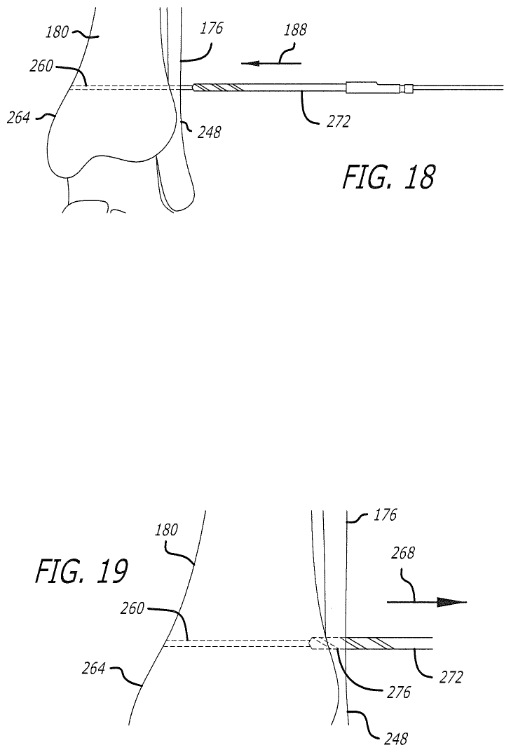

Once the guidewire 232 is desirably positioned across the first and second bones 176, 180, a first cannulated bone drill 272 may be moved in the distal direction 188 along the guidewire 232 to the near cortex 248 of the first bone 176, as shown in FIG. 18. As shown in FIG. 19, the first cannulated bone drill 272 may be used to drill a first bone hole 276 that extends through the first bone 176. Once the first cannulated bone drill 272 breaches a far cortex (not shown) of the first bone 176, the bone drill 272 may be moved in the proximal direction 268 and removed from the guidewire 232.

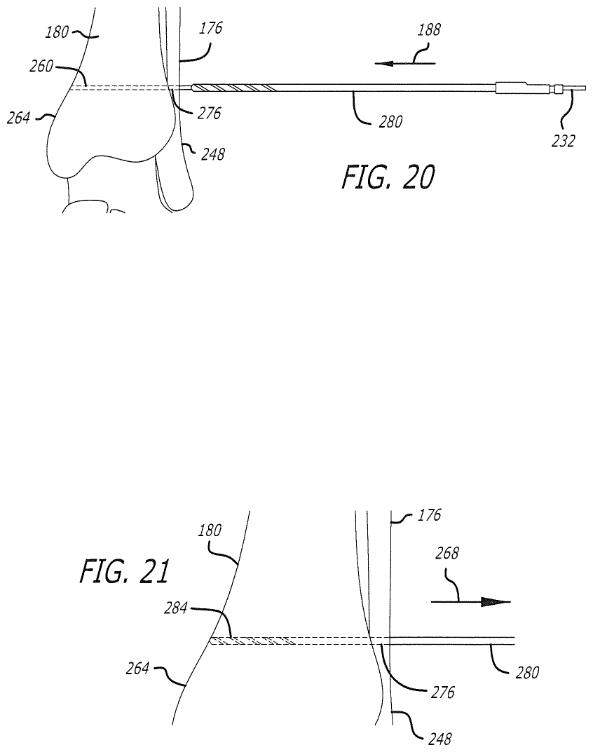

Next, a second cannulated bone drill 280 may be moved in the distal direction 188 along the guidewire 232 and into the first bone hole 276, as shown in FIG. 20. Preferably, the first cannulated bone drill 272 has a greater diameter than the second cannulated bone drill 280, such that the second cannulated bone drill 280 may be passed freely through the first bone hole 276. As shown in FIG. 21, the second cannulated bone drill 280 may be used to drill a second bone hole 284 through the second bone 180. Once the second cannulated bone drill 280 breaches the far cortex 264 of the second bone 180, the second cannulated bone drill 280 and the guidewire 232 may be moved in the proximal direction 268 and removed from the patient.

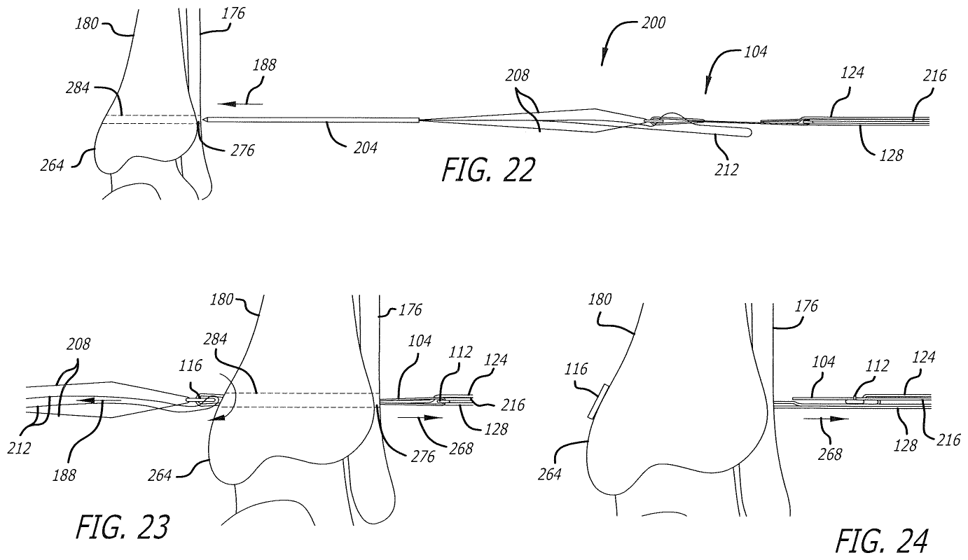

As best shown in FIG. 22, the syndesmosis treatment assembly 200 may be extended through the first and second bones 176, 180 by first inserting the deployment guide wire 204 into the first bone hole 276 and then advancing the deployment guide wire 204 in the distal direction 188. The deployment guide wire 204 may then be pushed through the second bone hole 284 until the distal end 224 of the deployment guide wire 204 breaches the skin adjacent to the far cortex 264 of the second bone 180. The deployment guide wire 204 and the pull wire 208 may be pulled in the distal direction 188 until the distal fixator 116 moves through the far cortex 264 and exits the second bone hole 284 of the second bone 180.

Once the syndesmosis treatment assembly 200 is optimally positioned with respect to the far cortex 264 of the second bone 180, as shown in FIG. 23, the distal fixator 116 may be deployed by pulling the deployment suture 212 in the distal direction 188. Movement of the syndesmosis treatment assembly 200 within the bone holes 276, 284 may be prevented by pulling the proximal deployment suture 216 in the proximal direction 268. Pulling the deployment suture 212 causes the distal fixator 116 to rotate into an orientation suitable for being placed into contact with the far cortex 264 of the second bone 180. Once the distal fixator 116 is optimally anchored against the far cortex 264 of the second bone 180, the pull wire 208 and the deployment suture 212 may be snipped and withdrawn from the distal fixator 116, as shown in FIG. 24, leaving only the syndesmosis suture construct 104 disposed within the first and second bones 176, 180.

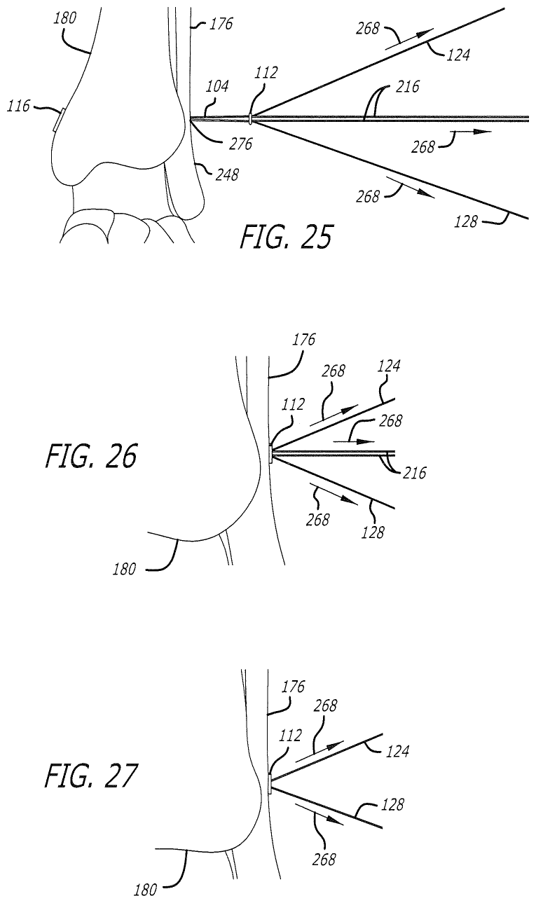

Next, the proximal fixator 112 may be anchored against the near cortex 248 of the first bone 176. As shown in FIG. 25, tension may be maintained on the proximal deployment suture 216 while the suture ends 124, 128 are pulled in the proximal direction 268 to tighten the syndesmosis suture construct 104 within the first and second bones 176, 180 and thus to cinch the first bone 176 against the second bone 180, as desired. The suture ends 124, 128 may be pulled by way of a tensioning handle 288, shown in FIG. 29, that ensures the sutures ends 124, 128 are exposed to substantially the same tension. During cinching the syndesmosis suture construct 104, the proximal fixator 112 seats within a proximal opening of the first bone hole 276, as shown in FIG. 26. In instances wherein a bone fusion plate 256 is pressed against the first bone 176, as shown in FIG. 16, cinching the syndesmosis suture construct 104 seats the proximal fixator 112 within the aperture 252 of the bone fusion plate 256. Once the bones 176, 180 are desirably cinched together, the proximal deployment suture 216 may be removed and removed from the proximal fixator 112, as shown in FIG. 27. Any one or more of various surgical knots may be formed by the surgeon to ensure that the syndesmosis suture construct 104 maintains a desirably cinched configuration of the first and second bones 176, 180.

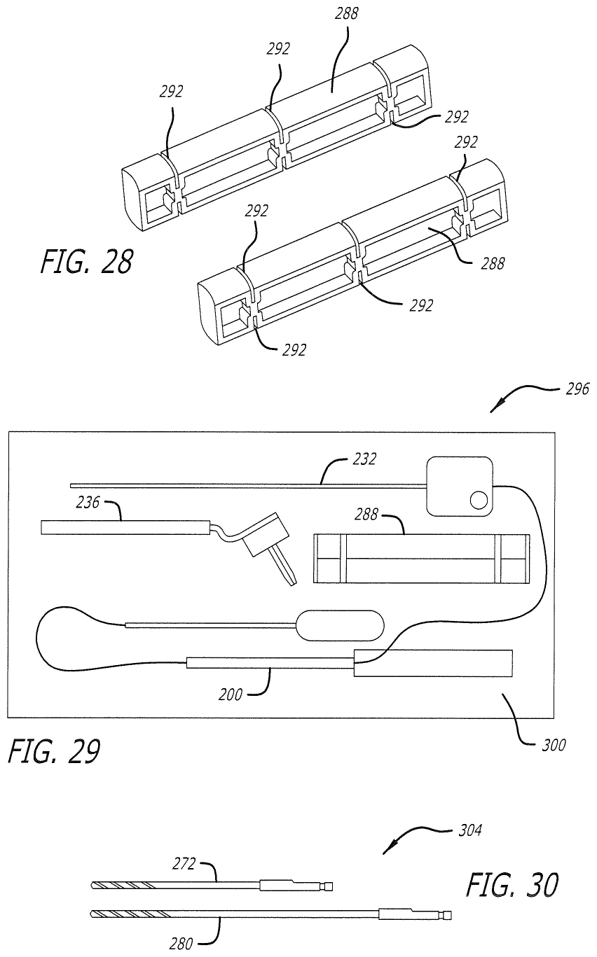

FIG. 28 illustrates an exemplary embodiment of a pair of tensioning handles 288 that are configured to facilitate pulling the sutures ends 124, 128 during cinching the first and second bones 176, 180 together. The tensioning handle 288 is a generally elongate member having suture grooves 292 disposed along the length of the tensioning handle 288. The sutures grooves 292 are each configured to fixedly receive a portion of suture such that a surgeon may grasp the tension handle 288 during pulling the suture. It is contemplated that the surgeon may use a first tensioning handle 288 to maintain tension on the proximal deployment suture 216 while using a second tensioning handle 288 to pull the suture ends 124, 128 simultaneously in the proximal direction 268, as described hereinabove. Although in the illustrated embodiment, three suture grooves 292 are uniformly disposed along each tension handle 288, any number of suture grooves 292 may be incorporated into the tension handles 288.

Turning, now, to FIG. 29, an exemplary embodiment of a syndesmosis implant kit 296 is shown for cinching a first bone against a second bone according to the present disclosure. In the embodiment illustrated in FIG. 29, the syndesmosis implant kit 296 includes an instrument tray 300 that holds at least a syndesmosis treatment assembly 200, a guidewire 232, a wire guide 236, and a pair of tensioning handles 288. In some embodiments, the syndesmosis implant kit 296 may further include a syndesmosis reamer kit 304 comprising at least a first cannulated bone drill 272 and a second cannulated bone drill 280, as shown in FIG. 30. As will be appreciated, the syndesmosis implant kit 296 comprises instruments necessary for treating syndesmosis injuries by way of surgery. The sizes of the instruments comprising the syndesmosis implant kit 296 will depend upon the size of the bones to be cinched. It is envisioned, therefore, that a surgeon may select the syndesmosis implant kit 296 and the syndesmosis reamer kit 304 based on the location and size of the bone joint to be treated.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

What is claimed is:

1. A syndesmosis treatment assembly, comprising:
a syndesmosis suture construct for securing a first bone against a second bone;
an insertion assembly for implanting the syndesmosis suture construct in a patient; and
one or more tensioning handles for cinching the syndesmosis suture construct.

2. The syndesmosis treatment assembly of claim 1, wherein the insertion assembly includes a deployment guidewire, a pull wire, a deployment suture, and a proximal deployment suture.

3. The syndesmosis treatment assembly of claim 1, wherein the deployment guidewire is a generally elongate member that includes a sharpened distal end suitable for being passed through a bone hole drilled into a first bone and a second bone of a patient.

4. The syndesmosis treatment assembly of claim 3, wherein the pull wire is attached to a proximal end of the deployment guidewire and coupled with a distal fixator comprising the syndesmosis suture construct.

5. The syndesmosis treatment assembly of claim 4, wherein the deployment guidewire, the pull wire, and the proximal deployment suture may be used to maintain the syndesmosis suture construct in an elongate configuration suitable for being inserted into the bone hole drilled.

6. The syndesmosis treatment assembly of claim 5, wherein the deployment suture is configured to enable a surgeon to cause the distal fixator of the syndesmosis suture construct to contact a far cortex of the second bone.

7. An insertion assembly for implanting a syndesmosis suture construct in a bone hole drilled across a first bone and a second bone of a patient, the insertion assembly comprising:
a deployment guidewire;
a pull wire;
a deployment suture; and
a proximal deployment suture.

8. The insertion assembly of claim 7, wherein the deployment guidewire is configured for inserting the syndesmosis suture construct into the bone hole.

9. The insertion assembly of claim 7, wherein the deployment guidewire includes a sharpened distal end for being passed through skin adjacent to a far cortex of the second bone.

10. The insertion assembly of claim 7, wherein the pull wire is configured for drawing a distal fixator comprising the syndesmosis suture construct through the bone hole.

11. The insertion assembly of claim 10, wherein the deployment suture is configured for deploying the distal fixator against a far cortex of the second bone.

12. The insertion assembly of claim 10, wherein the proximal deployment suture is configured for maintaining the syndesmosis suture construct in an elongate configuration suitable for being inserted into the bone hole.

13. The insertion assembly of claim 7, further comprising one or more tensioning handles configured to facilitate pulling suture ends comprising the syndesmosis suture construct during cinching the first bone and the second bone.

14. The insertion assembly of claim 13, wherein the one or more tensioning handles each comprises a generally elongate member having suture grooves disposed along a length of the tensioning handle.

15. The insertion assembly of claim 14, wherein the sutures grooves are each configured to fixedly receive a portion of suture such that a surgeon may grasp the tensioning handle during pulling the suture.

16. A method for treating a syndesmosis by securing a first bone and a second bone of a patient, comprising:
drilling a bone hole through the first bone and the second bone;
providing a syndesmosis suture construct for inserting into the bone hole and cinching the first bone against the second bone;
pulling the syndesmosis suture construct through the bone hole by way of a deployment guidewire and a pull wire;
deploying a distal fixator of the syndesmosis suture construct by way of a deployment suture;
maintaining tension on the syndesmosis suture construct by way of a proximal deployment suture;
cinching the syndesmosis suture construct to press the first bone against the second bone; and
forming one or more surgical knots to secure a cinched configuration of the first bone and the second bone.

17. The method of claim 16, wherein drilling includes using a first cannulated bone drill to form a first bone hole through the first bone and then using a second cannulated bone drill to form a second bone hole through the second bone, the first cannulated bone drill having a larger diameter than a diameter of the second cannulated bone drill.

18. The method of claim 16, wherein pulling the syndesmosis suture construct includes passing the deployment guidewire and the pull wire through skin near a far cortex of the second bone.

19. The method of claim 18, wherein deploying includes using the deployment suture to cause the distal fixator to contact the far cortex of the second bone.

20. The method of claim 19, wherein deploying includes removing the deployment guidewire, the pull wire, and the deployment suture after causing the distal fixator to contact the far cortex of the second bone.
